(19) United States
(12) Patent Application Publication
(10) Pub. No.: US 2012/0178051 A1
(43) Pub. Date: Jul. 12, 2012

(54) Anesthesia delivery system

(76) Inventor: William Monroe Stephenson, Slidell, LA (US)

(21) Appl. No.: 12/930,639

(22) Filed: Jan. 12, 2011

(51) Int. Cl. A61C 19/08; B82Y 5/00

(52) U.S. Cl. 433/215; 977/906

(57) ABSTRACT

An anesthesia delivery system comprising a nanosphere forming a bubble into which an anesthesia is introduced and delivered with a swab of absorbent material to a site to be treated.

### FIELD OF THE INVENTION

The present Invention relates to an anesthesia delivery system comprising a nanosphere forming a bubble into which an anesthesia is introduced and delivered with a cotton swab to a site to be treated. In one embodiment the anesthesia is Lidocaine, a synthetic amide, C 14 H 22 N 2 O, used chiefly in the form of its hydrochloride as a local anesthetic and antiarrhythmic agent.

### DESCRIPTION OF THE PRIOR ART

Anesthetics and anesthesia's are well known. However, delivery of same using a nanosphere carrier is not known.

### BRIEF SUMMARY OF THE INVENTION

According to the teachings of the present invention, nanospheres are filled with anesthesia in different percentages for delivery via a swab of absorbent material to a selected delivery site.

### DETAILED DESCRIPTION OF THE INVENTION

In use of the teachings of the invention, nanospheres, such as the ones obtained from are saturated with a dose of an anesthetic, such as a blend of anesthetyic and water. For example Dentists use 2 mg, 4 mg, and 6 mg doses of Lidocaine.

Nanospheres are saturated with:
A 30% anesthetic for a 6% dose.
A 20% anesthetic for a 4% dose
A 10% anesthetic for a 2% dose

Empirical tests show that delivery of nanospheres saturated with one of the doses set forth above to a specified area relieves pain in that area without numbing adjacent areas.

Ingredients in one preferred embodiment are:

| | % w/w | Vendor |
|---|---|---|
| DI Water | up to 100% | In-house |
| Glycerin 99% | 5.0% | Univar |
| Thixogel XLG | 2.0% | TCR |
| Sodium Saccharin | 0.25% | Univar |
| Polysorbate 80 | 1.0% | Univar |
| Flavor (Spearmint oil) | 0.25% | Integrity Ingredients |
| Citric Acid | 0.025% | Any |
| Potassium Sorbate | 0.15% | Any |
| Sodium Benzoate | 0.05% | Any |
| Lidocaine | 40.0% | Salvona |
| Alcohol 38B | 10.0% | Remet (Alcohol denatured |
| | | with menthol) |

Add the Thixogel to the water slowly, while mixing. Mix for 10 minutes. Add the Sodium Saccharin and mix for 5 minutes. Premix the flavor with the Polysorbate 80 and add to the product. Add all other ingredients in order.
Alternatives anesthesias known to date:

| | |
|---|---|
| Articaine: brand names: | septocaine and zorcaine |
| Mepivacaine: brand names: | carbocaine and polocaine |
| marcaine or bupivacaine | long lasting 6 hours. |

In one embodiment a dentist uses a 1/16 diameter pipe that fits on the end of the applicator instead of a needle. It is about ¾″ long and then makes a 90% bend and entends ¾″ to an opening of the pipe. The dentist can put this device on the end of a capsule filled with anesthesia saturated nanospheres and put a spot of it on the top of the tooth to be fixed. It is similar to using a hyperdermic except without the needle. The dentist uses the bent pipe instead of a needle. Also the dentist can apply the anesthesia saturated nanosheres to the gum area adjacent the tooth.

From the foregoing description, it will be apparent that the system of the present invention has a number of advantages, namely delivery of a sufficient quantity of antiseptic to a specific area to relieve pain without numbing adjacent areas. Accordingly the scope of the present invention is only to be limited by the accompanying claims.

1. An anesthesia delivery system comprising a nanosphere forming a bubble into which an anesthesia is introduced and delivered with a swab of absorbent material to a site to be treated.

2. The delivery system according to claim 1 wherein the anesthesia is a solution of Lidocaine and water.

3. The delivery system according to claim 1 wherein said anesthesia is a solution of:

| DI Water | up to 100% |
|---|---|
| Glycerin 99% | 5.0% |
| Thixogel XLG | 2.0% |
| Sodium Saccharin | 0.25% |
| Polysorbate 80 | 1.0% |
| Flavor (Spearmint oil) | 0.25% |
| Citric Acid | 0.025% |
| Potassium Sorbate | 0.15% |
| Sodium Benzoate | 0.05 |
| Lidocaine | 40.0% |
| Alcohol 38B | 10.0% |

4. The delivery system according to claim 1 wherein said anesthesia is septocaine and zorcaine.

5. The delivery system according to claim 1 wherein said anesthesia is carbocaine and polocaine.

6. The delivery system according to claim 1 wherein said anesthesia is marcaine.

7. The delivery system according to claim 1 wherein said anesthesia is bupivacaine.

8. The delivery system according to claim 1 wherein said anesthesia saturated nanospheres are placed on the top of a tooth to be treated.
